(19) United States
(12) Patent Application Publication
(10) Pub. No.: US 2007/0167724 A1
(43) Pub. Date: Jul. 19, 2007

(54) fMRI data acquisition system

(76) Inventors: Hrishikesh P. Gadagkar, Waukesha, WI (US); Catherine L. Elsinger, Wauwatosa, WI (US); Stephanie Sylve, Wauwatosa, WI (US); Stephen M. Rao, Shorewood, WI (US)

Correspondence Address: John Horn;Neurognostics, Inc., Suite 309, 10437 Innovation Drive, Milwaukee, WI, 53226, US

(21) Appl. No.: 11/298,876

(22) Filed: Dec. 9, 2005

(51) Int. Cl. A61B 5/05

(52) U.S. Cl. 600/410

(57) ABSTRACT

A data acquisition system for use in conducting functional magnetic resonance imaging studies. fMRI studies can provide clues to the neurobiological basis of CNS disorders and reliable and quantifiable data relating to their symptoms. The data acquisition system includes a control station, a patient stimulus and response system and fMRI data acquisition software for controlling the operation of the system in response to operator input and acquiring fMRI data comprising MR images and associated behavioral response data. The stimulus and response system presents visual or auditory or other stimuli to the patient under direction of the control station while functional MRI scanning takes place. The MRI image data is collected from the MRI scanner and combined with the behavioral data generated as the patient responds to the stimuli and archived for later analysis and use. The system includes quality control measures for properly setting up the equipment and preparing and training the patient and for verifying data quality during the different steps in the process.

### FIELD OF THE INVENTION

The present invention relates to systems for use in mapping the human brain and in detecting symptoms of neurological disorders and more specifically to the use of functional magnetic resonance imaging (fMRI) in pre-surgical mapping and in detecting symptoms, determining severity and assessing therapeutic efficacy in cases of central nervous system disorders.

### BACKGROUND OF THE INVENTION

fMRI is a neuroimaging technology which has been used in researching functional aspects of central nervous system (CNS) disorders. fMRI is an application of nuclear magnetic resonance technology in which functional brain activity is detected usually in response to a task specifically designed to evoke cognitive or motor behavior in a patient. fMRI is capable of detecting localized event-related brain activity and changes in this activity over time. Its principal advantages are its strong spatial and temporal resolution. Further, since no isotopes are used, a virtually unlimited number of scanning sessions that can be performed on a given subject, making within-subject studies feasible. fMRI operates by detecting increases in cerebral blood volume that occur locally in association with increased neuronal activity. A widely used fMRI method for detecting brain activity is based upon the blood oxygenation level dependent (BOLD) response. The BOLD signal arises as a consequence of a ‘paradoxical’ increase in blood oxygenation, presumably due to increased local blood flow in excess of local metabolic demand and oxygen consumption following neuronal activity. An increase in blood oxygenation results in increased field homogeneity (increase in T2 and T2*), less dephasing of spins, and increased MR signal intensity on susceptibility-weighted MRI images. fMRI systems can provide clues to the neurobiological basis of CNS disorders and reliable and quantifiable data relating to their symptoms. Accordingly, fMRI has been under increasing development as an instrument for assessing the neurobiological circuitry that underlies neurological disorders and for measuring the brain's response to therapeutic and especially pharmacological interventions. However, one of the main challenges of implementing fMRI technology has been the complexity associated with acquiring fMRI data and assembling, testing, configuring and maintaining the required fMRI equipment so that clinical evaluations of a variety of cognitive and sensorimotor functions can take place on an efficient and effective basis.

### SUMMARY OF THE INVENTION

The present invention comprises a data acquisition system for operating in conjunction with an MR scanner and acquiring fMRI data including MR images and associated behavioral response data. This system is comprised of two main components, a patient stimulus and response system, located in the MR scanner room, and a control station operating under control of a data acquisition software program, located in the MR control room. The stimulus and response system includes a presentation device for providing visual, auditory or other input to the patient such as a video projector and screen or a headphones and a patient response device for use by the patient in making responses such as a small keypad. The control station includes a workstation, the control application software and presentation, data collection and archiving software. The control station's software application program controls the operation of the patient stimulus and response system and coordinates the fMRI data acquisition process. Coordination of the data acquisition process includes the input of patient information, the selection of stimulation paradigms/activation tasks, the designation of scanning parameters, quality control mechanisms that track the function of all primary components in the system, data quality monitoring of patient responses, the collection of image data from the MR scanner and the formatting and archiving of all the data and information from the test session. The control station and its software guide the operator through the steps that make up an fMRI study and provide quality assurance procedures to assure that quality data is acquired.

The system is used as a data acquisition tool in the MR environment to perform functional MRI (fMRI) procedures based on Blood Oxygen Level Dependent (BOLD) contrast. The control station regulates the presentation of sets of stimuli to the patient by the stimulus and response system and the collection the patient's responses from a button (response) device in coordination with the operation of the MR scanner. Execution of specific cognitive or motor activation tasks by the patient in response to sets of specially selected stimuli that are synchronized in time with the acquisition of MR images creates the desired fMRI image data and behavioral data. The MR image data are obtained by the control station from the MR scanner console. The image and behavioral data are then combined and archived to a removable media.

The fMRI examination process is comprised of several steps. After the fMRI software application is launched the operator is prompted to input patient identification information. The operator then selects an activation task or stimulation paradigm appropriate for the patient, the disorder affecting the patient and purposes of the examination. At this stage the operator may engage a training mode whereby the patient is exposed to an emulated activation task reflecting the selected stimulation paradigm in order to familiarize the patient with task procedures. Thereafter, the operator is prompted to prepare (e.g. provide vision correction if necessary) and properly position the patient in the scanner, and verify proper equipment alignment and equipment configurations within the MR scanner environment. These procedures assure that the patient is able to see or hear the stimuli from the stimulus presentation equipment. Since proper operation of the response device is critical to the value of the fMRI data, the operator is guided through the process of checking the functionality of the response device prior to onset of the study. If all requirements have been met, the scan procedure can begin. First, an anatomical MR image of the patient's brain is then acquired with the MR scanner. The scanner operator checks and specifies the scan specifications for pulse sequences associated with high resolution anatomical images and lower resolution functional images. A subprogram is invoked for presenting a series of stimuli to the patient while MR scanning takes place for generating the functional MR images in coordination with the presentation of the stimuli. During functional scanning the responses of the patient are tracked and tallied and real time patient performance measures are presented to the operator. Additionally, a warning may be provided if the patient is not performing at minimum task performance levels. The MR image data is transferred from the MR scanner console to the control station and the integrity of the image data is verified. Finally, the image and associated behavioral data are stored in a standard format and archived onto a removable medium for later analysis and review.

It is an object of the present invention to provide an integrated fMRI data acquisition system including all primary hardware and software components for use in fMRI clinical applications.

It is an object of the present invention to provide an integrated fMRI data acquisition system for that is easily managed by an MR technologist and/or radiologist for developing and carrying out clinical applications.

It is an object of the present invention to provide an integrated fMRI data acquisition system including capabilities for synchronizing image acquisition with stimulus presentation, accessing behavioral performance measures, controlling quality through initiatives to minimize and correct head movement and testing response device and stimulus presentation equipment prior to study onset.

It is an object of the present invention to provide an integrated fMRI data acquisition system designed so that the MR technologist can be trained and guided through procedures for familiarizing the patient with activation tasks, positioning the patient so as to minimize head movement and monitoring the successful acquisition of behavioral and functional images for quality control purposes.

### BRIEF DESCRIPTION OF THE DRAWINGS

FIG. 1 is an overall diagrammatic view of an fMRI system including the MR scanner and the data acquisition system of the present invention.

FIG. 2 is a pictorial view of the hardware for the control station components of the data acquisition system of the present invention.

FIG. 3 is a pictorial view of the hardware for the projection and display components of the stimulus and response system of the data acquisition system of the present invention.

FIG. 4 is a pictorial view of the patient response device component of the stimulus and response system of the data acquisition system of the present invention.

FIG. 5 is a software flowchart showing the basic steps in the data acquisition program of the present invention run on the control station of the present invention.

FIG. 6 is a software flowchart showing sub-steps in the Prepare For MRI Scanning step of the present invention shown in FIG. 5.

FIG. 7 is a software flowchart showing sub-steps in the Perform MRI Imaging step of the present invention shown in FIG. 5.

FIG. 8 is a screen image of the configuration window associated with the Presentation application subprogram and data acquisition program of the present invention shown in this instance as active for input device configuration purposes.

FIG. 9 is a screen image of the Select Patient window for the data acquisition program of the present invention.

FIG. 10 is a screen image of the Select Task window for the data acquisition program of the present invention.

FIG. 11 is a screen image of the Patient Positioning window for the data acquisition program of the present invention.

FIGS. 12A is an image of an equipment setup assistance graphic included in the instructions presented to system operators in accordance with the present invention.

FIG. 12B is an image of an alignment screen presented to patients for use in aligning the components of the stimulus presentation system of the present invention.

FIG. 12C is an image of the patient response device presented to patients to assist in interactive equipment setup and verification in accordance with the present invention.

FIG. 12D is an image of a patient response device test window presented to system operators to assist in interactive equipment setup and verification in accordance with the present invention.

FIG. 13 is a screen image of the Training window for the data acquisition program of the present invention.

FIG. 14 is a screen image of the Anatomical Imaging window for the data acquisition program of the present invention.

FIG. 15 is a screen image of the functional Imaging window for the data acquisition program of the present invention.

FIG. 16A is a screen image of the main control window associated with the Presentation application subprogram and data acquisition program shown as active for initiating the running of a selected activation task.

FIG. 16B is a screen image of the activation task/stimulation paradigm/scenario selection window associated with the Presentation subprogram and data acquisition program that may be optionally used for making selecting new tasks or making changes to task selections.

FIG. 16C is a screen image of the activation task status and control windows associated with the Presentation application subprogram and data acquisition program of the present invention.

FIG. 16D is a screen image of a Data Integrity Failure window warning the operator that patient is not providing a minimum number of correct task responses provided for quality control purposes.

FIG. 17A is a screen image of the MR Image Transfer window for the data acquisition program of the present invention.

FIG. 17B is a screen image of a Dicom Image Error window warning that fewer files were transferred than expected provided for quality control purposes.

FIG. 18 is a screen image of the Study Comments window for the data acquisition program of the present invention.

FIG. 19 is a screen image of the Archive Patient Data window for the data acquisition program of the present invention.

FIG. 20 is a screen image of the data archiving window associated with the NovaBACKUP application subprogram and with the data acquisition program of the present invention shown as active for data archiving to a removable media.

### DETAILED DESCRIPTION

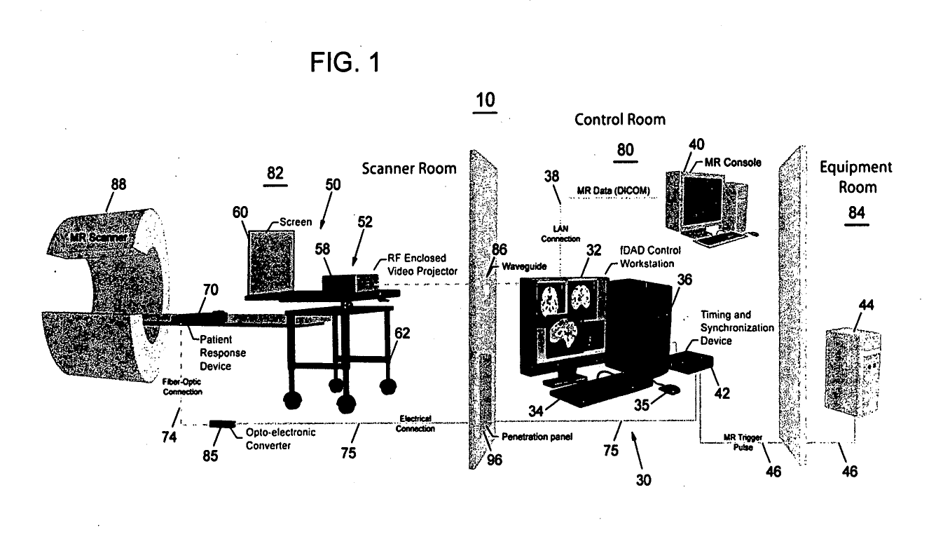

Referring now to FIG. 1, the data acquisition system 10 includes a control station 30 and a stimulus and response system 50. The control station 30 is located in the MR control room 80 and includes a 15 inch LCD flat panel monitor 32, input devices such as a keyboard 34 and mouse 35 and a control station computer (processor box) 36. The control station 30 comprises a computer workstation adapted to run a main data acquisition software application program for performing fMRI studies in accordance with the present invention. The application software includes certain auxiliary software programs (subprograms) which are adapted for running in conjunction with the main application program to provide otherwise conventional functions useful as a part of the main program. Stimulus presentation and response collection functions may be provided by a program such as the Presentation software program by Neurobehavioral Systems, Inc., 828 San Pablo Avenue, Suite 216, Albany, Calif. 94706. Data archiving functions may be provided by the program such as the NovaBACKUP software program by Novastor Corporation, 80B West Cochran, Simi Valley, Calif., 93065. Together with the main data acquisition program these programs constitute the primary software for controlling the data acquisition system and for providing the primary functionality of the present invention.

An Ethernet connection 38 is used to link the control station 30 to the MR scanner console 40 in the equipment room 84 for use collecting image data from the scanner. A timing, synchronization and interface device 42 is used to synchronize events related to the fMRI study including MR scanning, stimulus presentation and patient response collection and serves as an interface between the MR scanner system 44, the control station 30 and response device 70. The interface device 42 is connected to the control station computer 36 via an RS232 serial connection link. The interface device 42 provides a trigger pulse via an electrical or fiber optical connection 46 to the MR scanner system 44 and has a programmable feature to generate a triggered pulse train that has the same value as the TR (Repetition Time) for image acquisition, based on the values of the period, duration, and number of pulses needed for the fMRI study. The timing device 42 outputs the generated pulse train signal to the control station computer 36 via a RJ45 to parallel port computer link for use in stimulus timing. The control station software detects the rising edges of the pulse train and triggers stimulus presentation in accordance with the requirements for the fMRI study.

The stimulus and response system 50 is located in the scan room 82 comprises a video projection assembly 52 including a video projector, an optical receiver enclosed in an RF enclosure 58 and a display screen 60. The projection assembly 52 and screen 60 are both mounted on a mobile support cart 62 that allows vertical (or height) adjustment and horizontal movement to increase/decrease relative distance between the screen 60 and the projection assembly 52 and adjust the position of the screen 60 relative to the patient. A head coil (not shown) is preferably used with a mirror assembly designed to allow the patient to see the display screen 60 while reclined on the patient support table when inside the bore of the scanner magnet assembly 88. Alternatively, prism glasses may also be employed for enabling the patient to view the screen 60. The control station 30 controls the projection assembly 52 via an optical communications link comprising an electrical-to-optical converter and transmitter unit (not shown) connected to a video card installed on the control station computer 36. This optical link passes through the waveguide 86 and transmits the video signals from the control station 30 to the video projection assembly 52 for projection on the screen 60.

The response device 70 operates using optical technology to avoid electromagnetic interference and includes optical switches designed specifically for use in the MRI environment. The response device 70 consists of a small metal-free keypad containing four elongated keys or buttons designed for easy use by the patient. In use the response device 70 is usually placed on the lap of the patient for the length of the fMRI procedure. The response device 70 is connected to an opto-electronic converter 85, located in the control room 82, via a fiber optic cable 74. The converter 85 translates the optical signals into electrical signals and transmits these electrical signals to the electronic interface unit 42 on electrical signal line 75 which passes from the scanner room 82 into the control room 80 through a connector at the penetration panel 96. The interface unit 42 includes a controller that may be programmed to map the keypad buttons to specific ascii characters that are furnished to the control station computer 36 via a serial port connection.

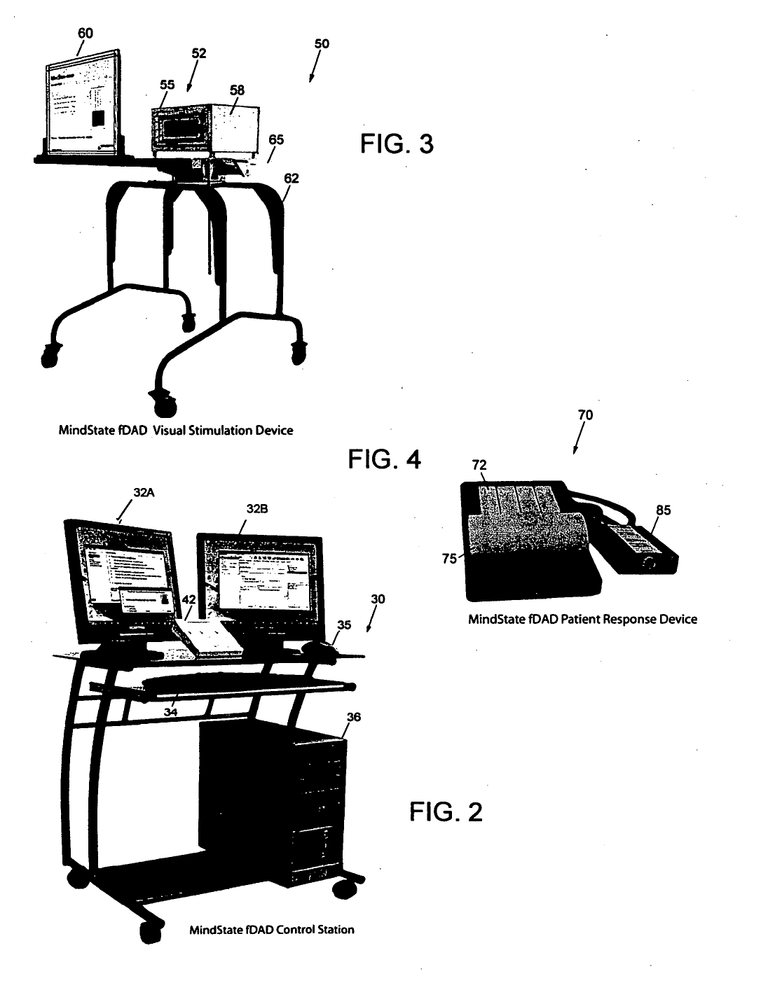

Referring now to FIG. 2, the control station 30 is shown in greater detail. In this case the control station 30 includes the two LCD monitors 32A and 32B for simultaneously monitoring both the operation of the data acquisition software application and the stimulation paradigm (although two monitors are not required). The control station also includes the interface unit 42, keyboard 34, mouse 35 and processor box 36. The control station 30 may comprise a 3 GHz microprocessor system running the Windows XP operating system by Microsoft and having 512 MB RAM, a 40 GB hard drive, a DVD+RW optical drive and a SVGA Video card such as a NVIDIA GeForce4 MX 4000 (in this case, a dual-display card).

Referring now to FIG. 3, the projection and display components of stimulus and response system 50 are shown as supported on a specially designed adjustable cart 62 adapted for being easily moved into position over a reclining patient. The projector assembly 52 is housed in the enclosure 58 and for shielding from electromagnetic interference. Images are projected onto the screen 60 through a small optical port in the door 55. The height of the projection components may be adjusted using a crank 65 operating a scissors jack supporting the table on which the system components are mounted.

Referring now to FIG. 4, the patient response components of the stimulus and response system 50 are shown. The response device 70 includes a keypad 72 having four elongated buttons or keys featuring shallow depressions for guiding the patient's fingers onto the correct keys and a pad 75 on which the patient's hand can rest for comfort and to help align the hand with the keypad 72. The optical-to-electrical interface unit 85 converts the optical signals generated by the response device 70 into standard electrical signals for use by the interface unit 42.

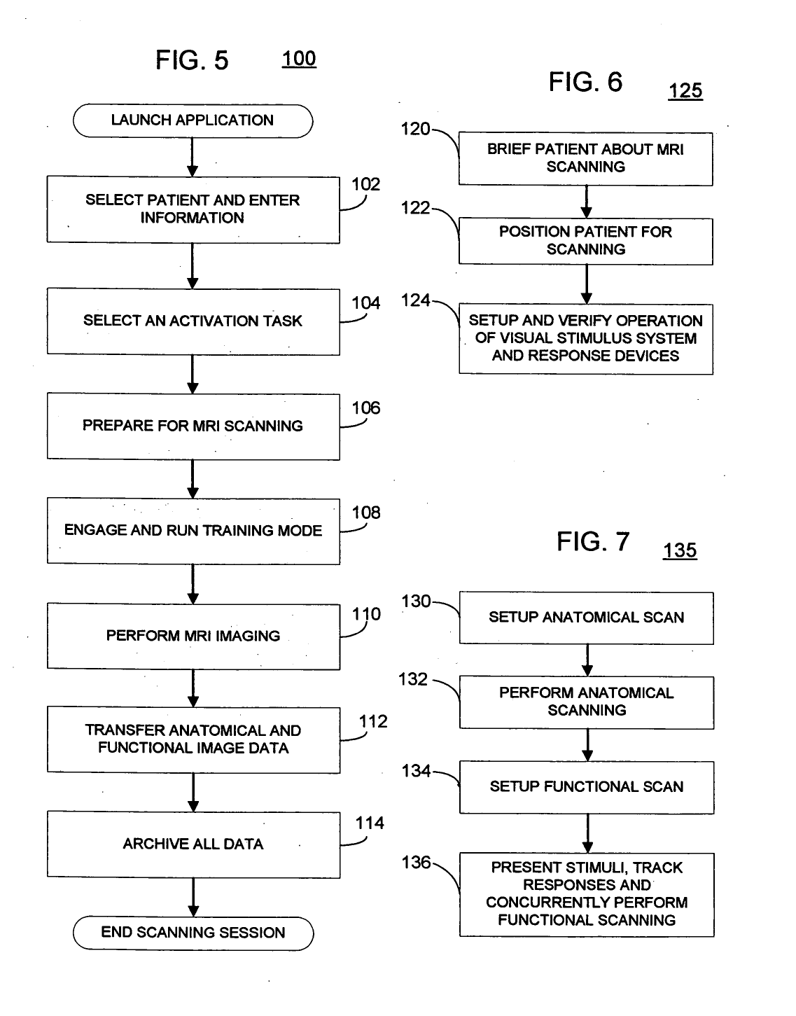

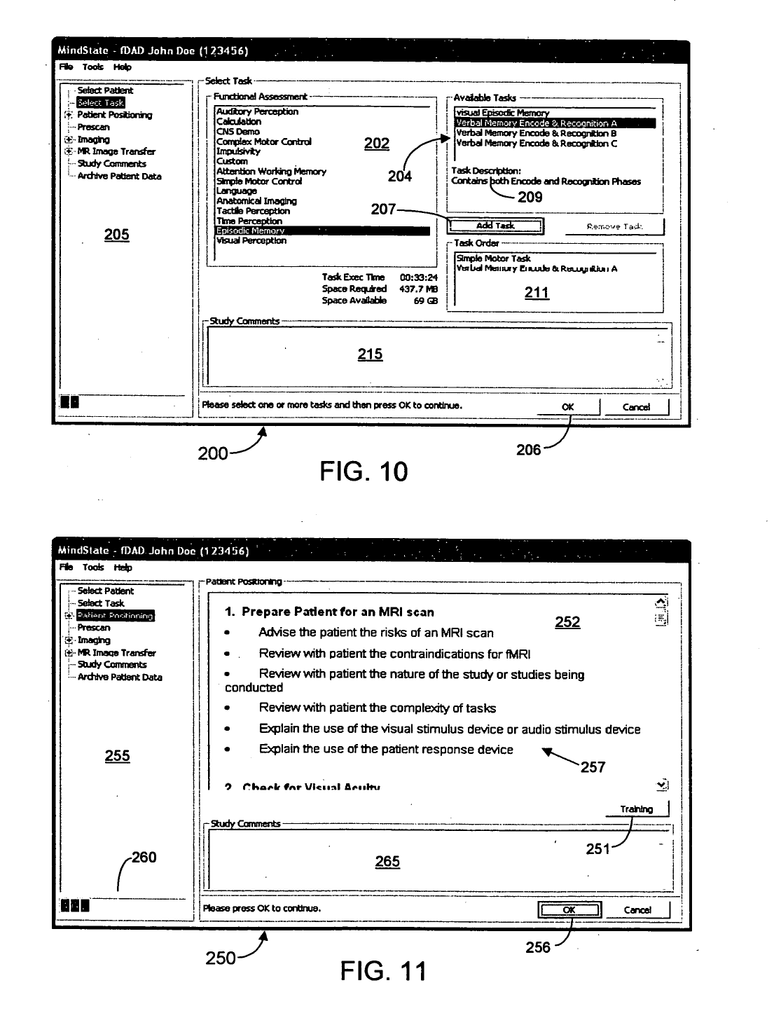

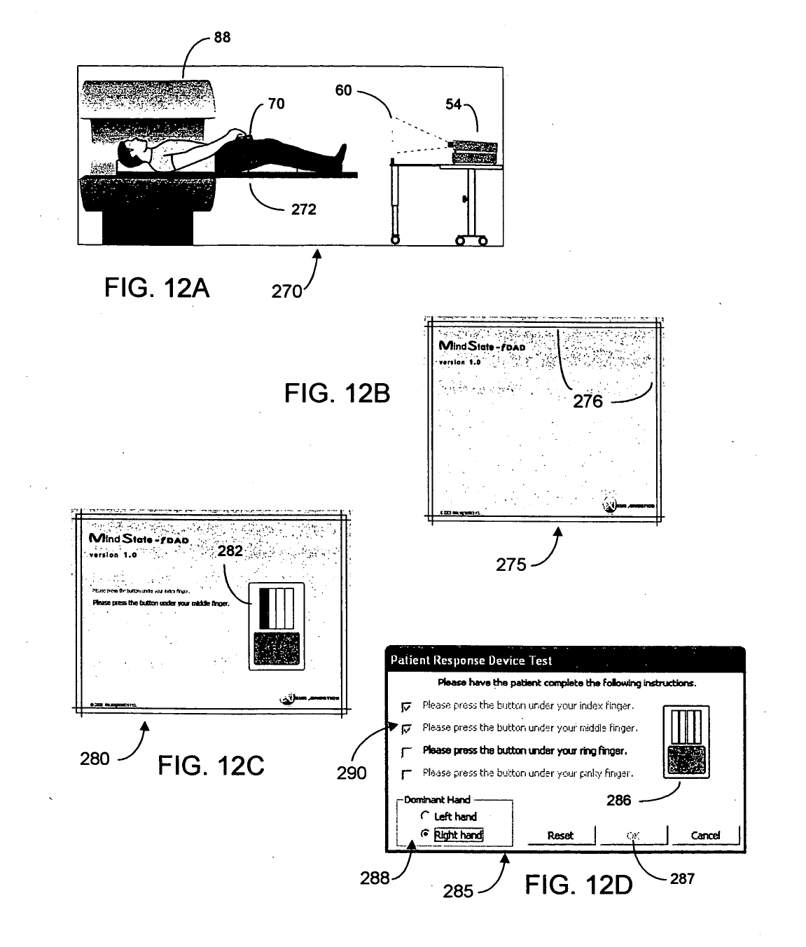

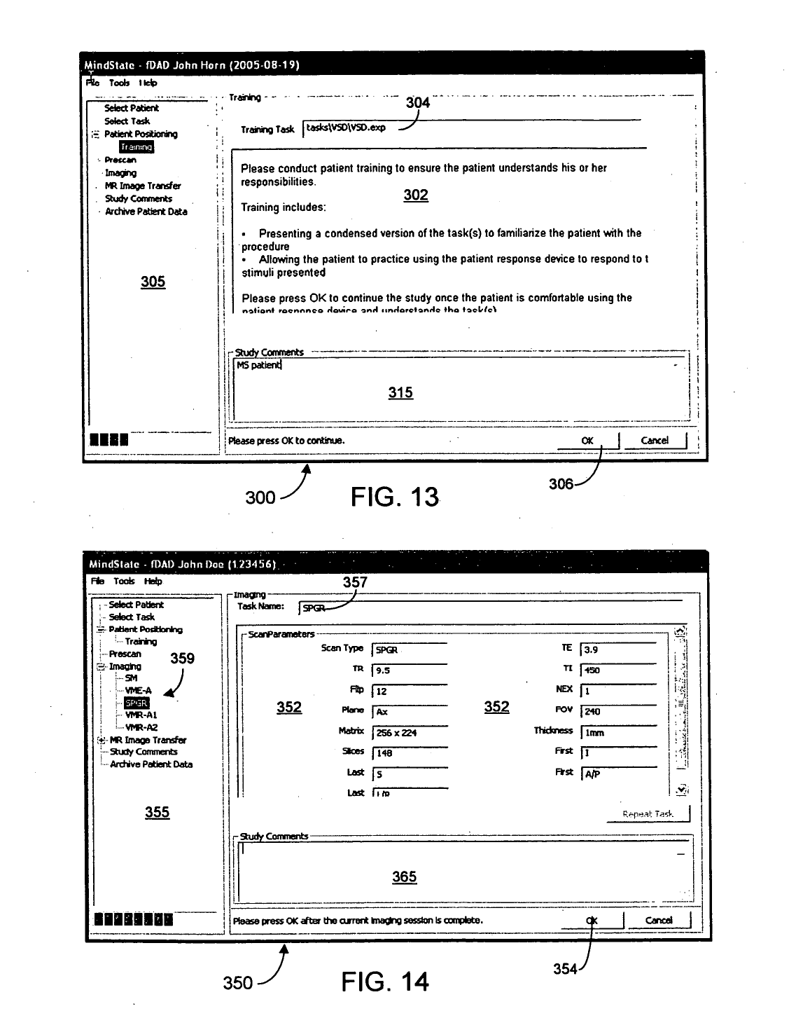

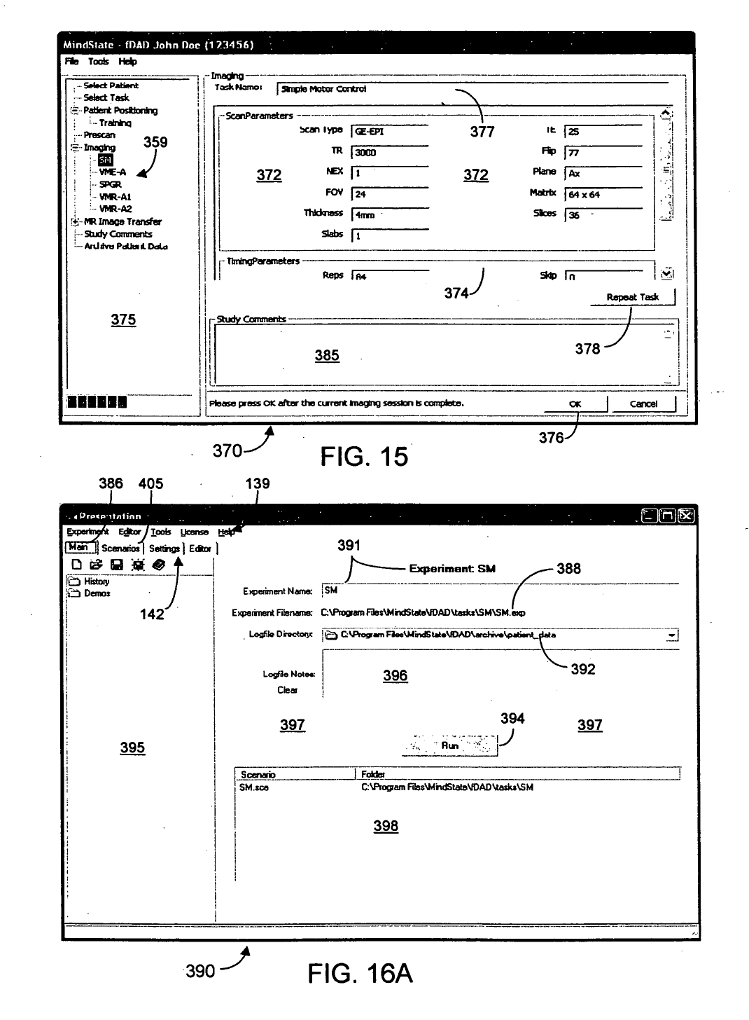

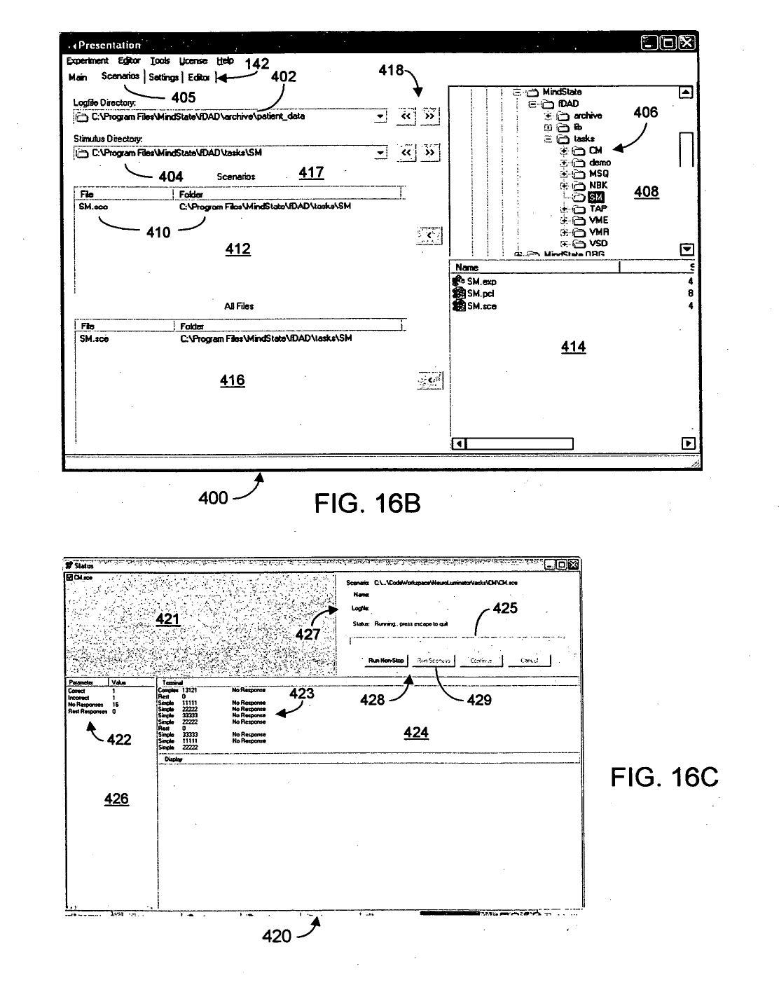

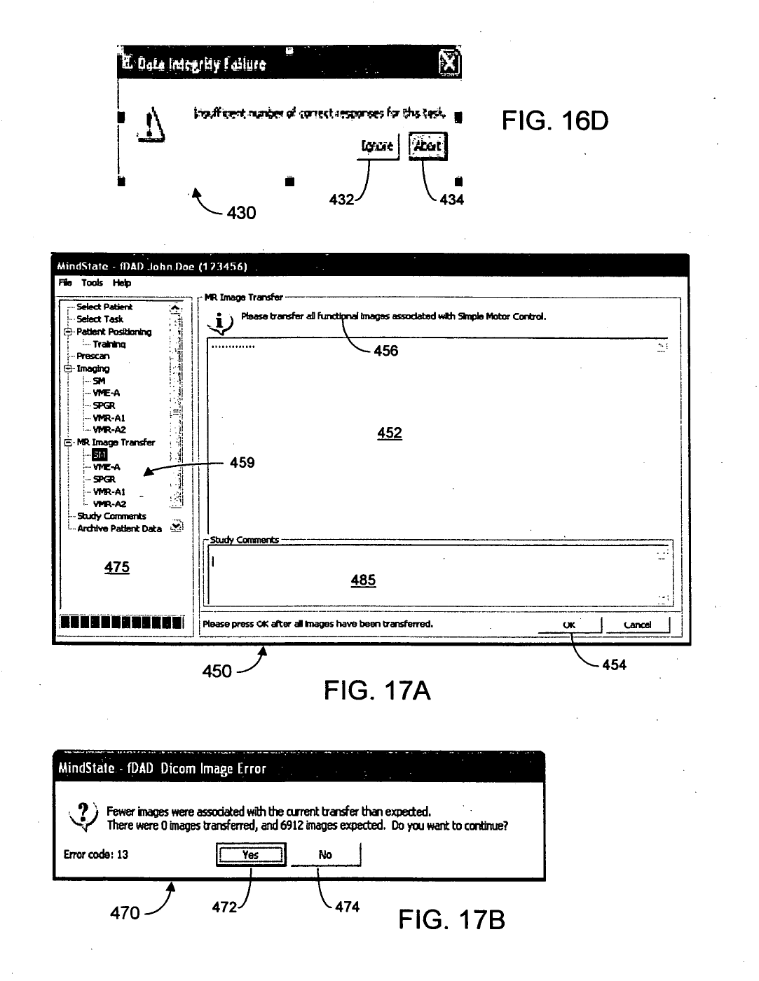

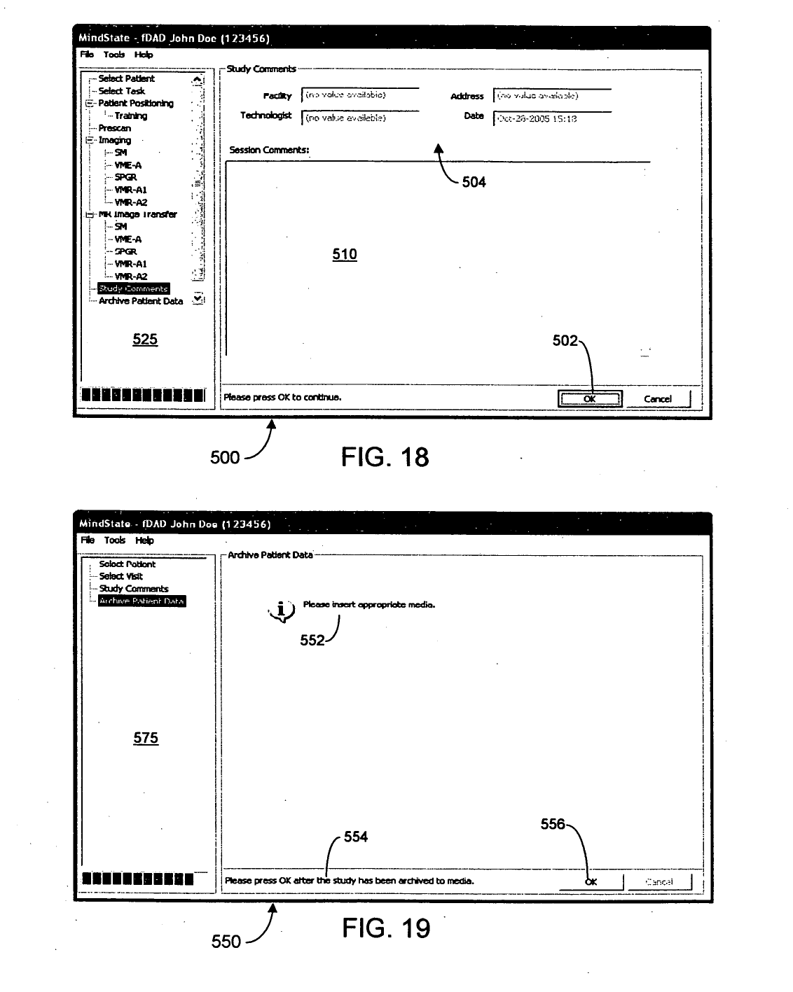

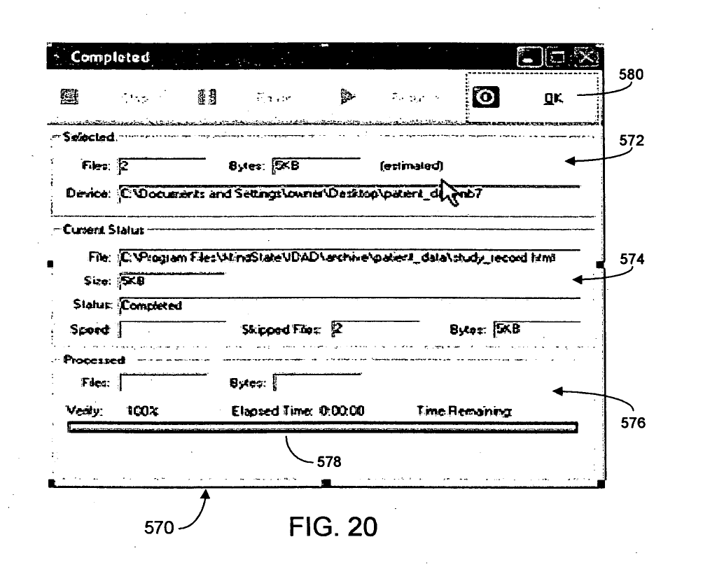

Referring now to FIG. 5, the flowchart 100 shows the operational process for the data acquisition system 10 as including seven basic steps: patient selection and patient information input 102, activation task/stimulation paradigm selection 104, execution of pre-scanning procedures 106, engaging and running a task training mode with the patient 108, execution of anatomical and functional imaging in conjunction with the performance of activation tasks by the patient 110, transfer of imaging data from the MRI scanner system 40 to the data acquisition system 112 and archiving of all collected data and comments to a removable media 114. These basic steps are performed through the use of and with the assistance of different interface screens: screen 150 in FIG. 9, screen 200 in FIG. 10, screen 250 in FIG. 11 (and related screens and windows 270, 275, 280 and 285 FIGS. 12A-12D), screen 300 in FIG. 13, screens 350 and 370 in FIGS. 14 and 15, screen 390 in FIG. 16A (and related screens 400, 420 and 430 in FIGS. 16B, 16C and 16D), screen 450 in FIG. 17A (and related screen 470 in FIG. 17B), screen 500 in FIG. 18 and screens 550 and 570 in FIGS. 19 and 20. The functions provided by the steps 102, 104, 106, 108, 110, 112 and 114 will be illustrated and described in conjunction with the interface screens shown in FIGS. 9-20 by means of which the functionality of these steps is implemented.

Referring now to FIG. 6 and 7, the flowcharts 125 and 135 expand on steps 106 and 110 in FIG. 5 and include substeps 120, 122 and 124 and substeps 130, 132, 134 and 136, respectively. These steps will be described in greater detail with respect to the screens and windows shown in FIGS. 12A-12D and FIGS. 14 and 15 with respect to which the functionality of these substeps is implemented.

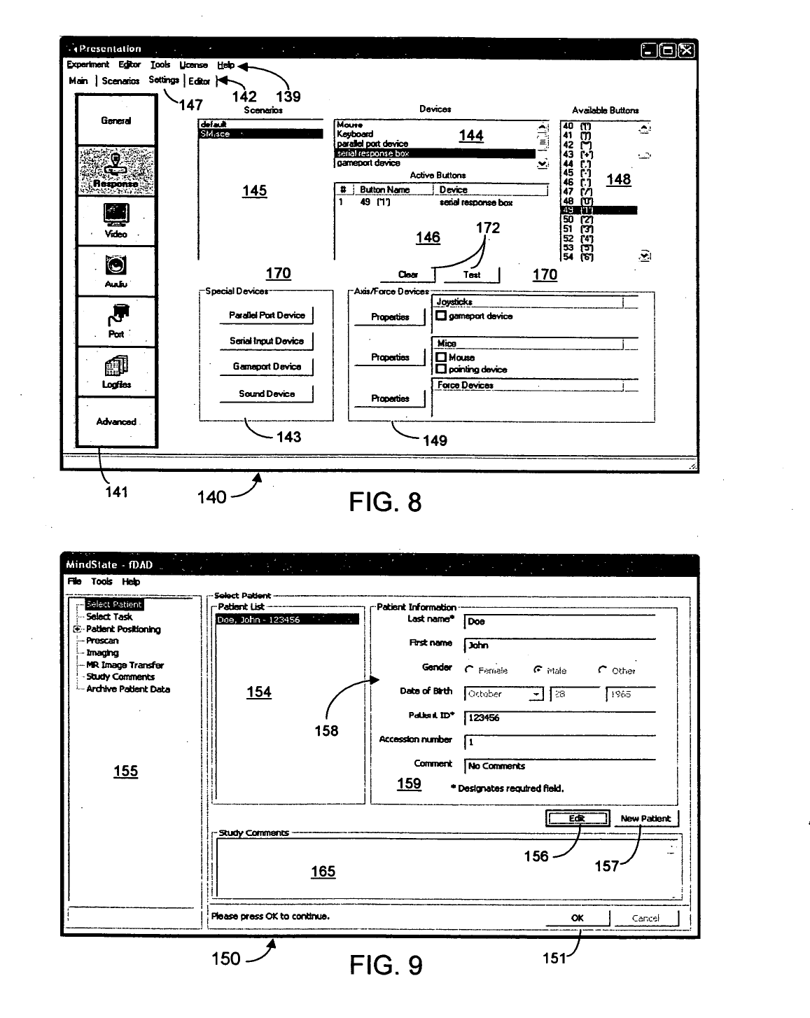

Referring now to FIG. 8, the Presentation application provides a configuration and control screen 140 in conjunction with the main data acquisition application that serves as an interface for setting up the hardware associated with the data acquisition system 10 and configuring the hardware for operation with the software applications and other equipment. The configuration and control screen 140 can be invoked at any time by clicking the presentation icon on the screen 32 of control station 30 and selecting the Settings tab 147 from the file tab set 142. Setup and configuration proceeds by selecting one of the buttons from the button bar 141 featuring a specific device or function and then entering information appropriate to the feature being configured on the settings panel 170. The buttons on the button bar 141 provide convenient access to configuration functions for selecting and configuring input/response devices, communications ports, video display adapters, audio equipment, log files and other system features. Required information is entered by clicking buttons, selecting list entries and entering alphanumeric text in text boxes and fields where appropriate in accordance with configuration panels provided in response to individual button selections.

Screen 140 specifically illustrates the set up process for a keypad input device (serial response box) such as the patient response device 70 having input buttons selected as a list entries in box 144 and configuring the keys of the device. Since response device configurations can be different for different types of tasks a task scenario must first be specified in box 145. With the system running and the software program launched the keys on the input device are pressed and are highlighted on the list in entry box 148. The keys may then be clicked on to enter them as active buttons on the list in entry box 146. The buttons 172 are used in testing the configurations. Special commonly used devices may be selected for configuration by general category using the buttons provided on panel 143 and the properties of selected devices may be readily accessed using the buttons on panel 149.

In normal operation, the data acquisition application software is launched by clicking a main fMRI data acquisition system application icon located on display 32 of the control station 30. After the data acquisition application software is first launched the operator is presented with a standard login screen and must provide a valid login name and password that must be authenticated for the operator to begin using the system 10. Thereafter, the operator is presented with the interface screens associated with the main program (and subprograms when their functions are required) and controls the operation of the system 10 by interacting with these screens to undertake and complete fMRI scanning study sessions. The application provides three pull down menus that are universally available and appear in every main screen at the top left corner of the windows, namely the File, Tools and Help menus. The Tools pull down menu provides access to DICOM related communications parameters such as port number that can be configured for a particular site by selecting a Customize option. The help menu provides an index of help topics including commentary although certain Help menu options are keyed to certain process steps and may be presented to and made conveniently available to the operator at certain times or upon selected events during the fMRI procedure. Help information may be accessed in the conventional manner by selecting the Help Contents option on the Help menu, examining the available topics and selecting the topic with respect to which assistance is required. The Help menu also provides a link to a web site providing product support. The operator may log out or exit the program at any time by selecting the File menu and clicking the Logout or Exit selections. Additional menus providing added functional options are provided during different process steps.

Referring now to FIG. 9, the Select Patient screen 150 associated with step 102 includes a set of text entry fields 158 on the Patient information panel 159 for the operator to add or edit patient information such as name, address and patient ID, although these fields may be automatically by populated with the necessary data by clicking the names of patients which appear on the Patient List box 154 for patients whose information has previously been entered into the system. The fields 158 may be made active for editing existing information or adding new patient information by clicking buttons 156 or 157. The screen 150 also includes a standard process steps box 155 common to most of the interface screens specifying the basic processing steps in the fMRI data acquisition process and highlighting the process step currently being performed so the operator has a visual cue as to the current step that is underway. The screen 150 further includes a standard text entry study comments box 165 common to most of the interface screens for use in entering miscellaneous information or commentary pertaining to the equipment, patient or procedure. After patient information is verified or new information is entered the operator proceeds to the next step in the procedure by clicking the OK button 151.

Referring now to FIG. 10, the Select Task screen 200 associated with step 104 includes a functional Assessment list box 202 providing a list of clinical assessments/indicated patient disorders that may be selected by the operator. In response to the selection of a particular assessment/disorder an adjacent Available Tasks list box 204 is populated with activation tasks/stimulation paradigms useful in fMRI studies related to the selected disorder. The operator may select one or more activation tasks/stimulation paradigms for the current patient and procedure by highlighting them in the box 204 and clicking the Add Task button 207 whereupon the tasks are listed in order of selection in the Task Order box 211. The screen 150 also includes a standard process steps box 205 (similar to box 155 in FIG. 9) listing the basic fMRI process steps and highlighting the current step to provide a visual cue as to the step currently underway and includes a standard text entry comment box 215 (similar to box 165 in FIG. 9) for entering miscellaneous information or commentary. After the activation task or tasks are selected the operator proceeds to the next step in the procedure by clicking the OK button 206.

Referring now to FIG. 11, the Patient Positioning screen 250 associated with step 106 includes a large Patient Positioning text box 252 for presenting a series of patient preparation and equipment setup instructions and graphics 257 to the operator in conjunction with the presentation of setup and instructional displays to the patient. The screen 250 also includes a standard process steps box 255 listing the basic fMRI process steps and highlighting the current step to provide a visual cue as to the step currently underway and includes a standard text entry comment box 265 for entering miscellaneous information or commentary. The instructions 257 provide for the operator to brief the patient on fMRI processes and advise the patient regarding risks associated with MRI scanning. The operator is also prompted to review with the patient the nature of the study, complexity of the tasks and explain the use of visual stimulation and patient response apparatus. The operator is then directed to prepare the patient for an MRI scan, help the patient in assuming a supine position, position any required head restraint system and instruct the patient to remain still. The operator is then directed to set up, position and align the visual stimulus device in front or behind the scanner table and patient response device under the patient's hand, and to complete setup verifications. A progress bar 260 (common to most of the interface screens) provides feedback to the operator as to degree to which the process step underway has been completed. As explained later a training procedure may be invoked as an optional procedure by clicking the Training button 251. After patient positioning, patent instruction, equipment setup and alignment and any desired patient training is completed, the operator proceeds to the next step in the procedure by clicking the OK button 256.

Referring now to FIGS. 12A and 12B, the graphical display image 270 is included within the instructions 257 provided to the operator and helps in directing the operator for proper setup and alignment of the projector assembly 54, display screen 60, response device 70 with respect to the patient 272 and scanner magnet 88. The display image 275 is presented on the screen 60 to the patient to insure that the visual stimulus system 50 is properly functioning and a complete test pattern as outlined by the border 276 is visible to the patient and the text on the screen 60 is legible to the patient. The stimulus and response system 50 can be adjusted in the vertical direction to move the screen up/down and in the horizontal direction to increase/decrease the relative distance between the screen and the assembly and can be moved towards or away from the patient as needed to provide that the patient is able to view the entire test pattern.

Referring now to FIGS. 12C and 12D, the interactive display screen 280 is also presented to the patient while the window 285 is displayed to the operator. The display screen 280 includes a test image 282 of the response device 70 on which the buttons are highlighted when pressed by the patient while the window 285 includes a similar test image 286 on which the buttons are also highlighted when pressed. The window 285 also includes radio buttons 288 for setting the handedness of the device 70 and check boxes 290 for use in verifying the functionality of each button. The display screen 280 and window 285 allow the operation of the response device 70 to be tested. After the procedures associated with step 106 and screen 250 are completed the operator clicks the OK button 256 to proceed to the next process step.

Referring now to FIG. 13, the Training screen 300 associated with step 108 includes a large text box 302 for presenting training instructions and graphics to the operator in conjunction with the presentation of a series of training displays and sample tasks and task elements to the patient on the patient display screen 60. The selected training task file location and name are displayed at field 304. The screen 300 also includes a standard process steps box 305 listing the basic fMRI process steps and highlighting the current step to provide a visual cue as to the step currently underway and includes a standard text entry comment box 315 for entering miscellaneous information or commentary. In operation it is critical to have the patient practice the activation task or tasks they will be performing in the scanner. Not only is it important for patient comfort and confidence in the context of the study, but it also ensures that the patient has a complete understanding of what they are expected to do and are able to perform at accuracy levels appropriate for the experiment. The training mode may be engaged from the Patient Positioning screen 250 by clicking the Training button 251. At this time display screen 280 and window 285 shown in FIGS. 12C and 12D may again be displayed to patient and operator to verify the operation of the patient response device and help familiarize the patient with operation of the buttons. At this point when the operator clicks the OK button 306 the data acquisition application will launch the Presentation program in a training mode version using the task selected previously while instructions are providing in the box 302 for conducting patient training. This also gives the operator an opportunity to ascertain whether the patient is able to respond correctly and within the allocated time for proper responses.

Following patient positioning, preparation and training, the data acquisition application displays a Pre-scan Operations screen (not shown in the Figures or referenced in flowchart 100) simply reminding the operator to perform your any unique pre-scan operations specific to the scanner equipment or required by the specific medical site and prompting the operator to click OK once these site-oriented pre-scan operations are complete.

Referring now to FIGS. 14 and 15, the Imaging screens 350 and 370 associated with process step 110 include sets of settings parameter fields 352 and 372 and timing parameter fields 374 for verifying scanning parameters for anatomical images and scanning and timing parameters for functional images according to the selected image (anatomical) or selected activation task indicating a functional image type. The screens 350 and 370 also include standard process steps boxes 355 and 375 and listing the basic fMRI process steps and highlighting the current step to provide a visual cue as to the step currently underway and include standard text entry comment boxes 365 and 385 for entering miscellaneous information or commentary. Available anatomical images and selected tasks representing functional images are shown on the list 359 in both box 355 and box 375 with the selected and highlighted image or task then also indicated in fields 357 and 377. After the anatomical imaging parameters (e.g. anatomical image: “SPGR”) are verified or modified, the OK button 354 on screen 350 is clicked to engage anatomical imaging. After task determined functional imaging parameters (e.g. activation task: “SM”) are verified or modified the OK button 376 on screen 370 is clicked to engage functional imaging and the Presentation subprogram application is launched. In the case of multiple functional tasks as shown functional imaging proceeds task-by-task according to the list 359 showing the selected tasks and the order in which the tasks are selected by the operator. During imaging functional tasks may be repeated as necessary by clicking the Repeat Task button 378.

Referring now to FIGS. 16A-16D, the Presentation application screen 390 (see also screen 140 in FIG. 8) appears after the Presentation subprogram for presenting stimuli to the patient is launched and includes a Main tab 386 in the file tab set 142 that provides information pertaining to the Presentation program and to the selected activation task on the main panel 397. The experiment or activation task name, the experiment filename and file location, and the log-file directory location for the selected activation task are indicated at fields 391, 388 and 392. Background information relating to the files is provided in text boxes 395, 396 and 398. The operator may engage the selected activation task under the Presentation subprogram at any time by clicking the Run button 394. Activation tasks are pre-configured for running under the Presentation sub-program, however, if the operator wishes to switch or change tasks, he or she may click the Scenarios tab 405 in file tab set 142 which opens a window for screen 400 (FIG. 16B) having a scenarios panel 417 including fields and text boxes enabling the selection different activation task files and folders. Panel 417 includes fields 402 and 404 indicating the data Logfile Directory and Stimulus (activation task) Directory. The Panel 417 also includes the Scenarios text box 412 indicating the file name and folder of the currently selected Scenario (or activation task) while the All Files text box 416 indicates the file names and folders of other selected data files pertaining to the current study. The directories, files and folders shown in fields 402 and 404 and text boxes 412 and 416 may be selected and changed by the operator by selecting from the files appearing in the file structure 406 shown in box 408 and the file list shown box 414 on the right side of the scenarios panel 417 using the selection buttons 418. The files and folders of any newly selected task or tasks or data files then show up in the fields and text boxes on the main panel 397 (invoked by the main tab 386) to be run by the Presentation subprogram. When the Run button 394 is clicked the window for the Status screen 420 (FIG. 16C) is opened. The Status screen 420 includes the text box 421 and the fields 427 specifying the selected scenario (activation task) file, scenario file name and folder, data log file and scenario status and includes buttons 428 for initiating the execution of the scenario (activation task). The operator may initiate a scenario by clicking the button 429. After a scenario (activation task) begins running the progress bar 425 provides a visual indication of the extent to which the scenario has been completed. Information about the actual task elements being presented to the patient and the nature of the patient's responses to those task elements is provided to the operator on a real time basis as a part of the task data listings 423 within the text box 424. Likewise, compiled data 422 is provided in real time in text box 426 indicating the ongoing number of correct, incorrect, no and rest responses by the patient. The data listings 423 and compiled patient response information 422 provide the operator with valuable real time information about how well the study is going so changes can be effected if required to adjust study conditions or interact with the patient. In the event the number of correct responses by the patient falls below a minimum level the Data Integrity Failure screen 430 (FIG. 16D) is automatically invoked by the program to inform the operator that the quality of the study is not meeting minimum standards. The operator may click the Ignore button 432 to proceed regardless of the warning or click the Abort to end the execution of the current activation task. After all activation tasks have been run the operator may close the Presentation subprogram.

Referring now to FIG. 17A and 17B, the MR Image Transfer screen 450 associated with step 112 provides the interface for the image transfer process to the fDAD control station 30 from the MRI scanner console 40. After stimulus presentation is complete, and all patient responses are collected and the Presentation subprogram is closed, the MR Image Transfer screen 450 is opened and the data acquisition application prompts the operator at the message field 456 to begin the upload the anatomical and the functional MRI image data. The operator highlights and clicks on the anatomical image (e.g. SPGR) file name or activation task name (e.g. SM) to initiate the transfer of anatomical or functional image data. Over the course of the transfer process information about the transfer is furnished to the operator in the message box 452 and the progress bar 458 tracks the extent to which the transfer process is complete. The screen 450 also includes a standard process steps box 475 listing the basic fMRI process steps and highlighting the current step to provide a visual cue as to the step currently underway and includes a standard text entry comment box 485 for entering miscellaneous information or commentary. After each set of anatomical and functional images is transferred the application will then check whether the expected number of image files have been transferred and if this is not the case a Dicom Image Error window 470 will appear providing a quality assurance warning and prompting the operator to click the yes button 472 to continue or the no button 474 to investigate or repeat the last image transfer procedure. After the final set of images is transferred the operator is prompted to confirm that the process is complete, and that the application program should move on to its next step by clicking the OK button 454. Referring now to FIG. 18, the study comments screen 500 (not referenced in flowchart 100) facilitates the final entry of comments by the operator. Following successful transfer of all anatomical and functional MR image data, the application invokes the study comments screen 500 and the data acquisition application thereby prompts the operator to enter any further comments the operator may have concerning the current fMRI study in comment box 510. The screen 500 also includes a standard process steps box 525 listing the basic fMRI process steps and highlighting the current step in order to provide a visual cue as to the step currently underway. When the operator is finished entering comments he or she clicks the OK button 502 to confirm that the comments are complete and the application passes on to the archiving step.

Referring now to FIGS. 19 and 20, archive patient data screen 550 associated with step 114 provides the initial interface for archiving the collected data to a removable media such as a CDROM or DVD. The operator is prompted by the message field 552 to insert a blank recording media into a disk recording drive and by the message field 554 to click the OK button 556 after the archiving process is complete. After the media is inserted the NovaBACKUP application is launched bringing up the archiving status screen 570. The anatomical and functional MR images along with the behavioral data and comments are compressed and archived onto the removable media inserted in the drive by operation of the NovaBACKUP application. Information relating to the archiving process is provided in the Selected fields 572, Current Status fields 574 and Processed fields 576. The progress bar 578 provides a visual indication to the operator of the extent to which the archiving process is complete. The control bar 580 allows the operator to control the recording process, if needed. The status fields 574 indicate when the transfer and verification process is complete. When the archiving process is complete the media tray is ejected from the drive, the NovaBACKUP application is terminated and the status screen 570 closed. When the operator clicks the OK button 556 on screen 550 the current fMRI study session is indicated as being over and the control station is returned to the Select Patient screen 150. The removable media provides the results of the study session for later use and analysis.

Although the invention has been described with reference to certain embodiments for which many implementation details have been described, it should be recognized that there are other embodiments within the spirit and scope of the claims and the invention is not intended to be limited by the details described or limited to the embodiments specifically disclosed.

1. A process for acquiring task-activated fMRI patient data using an MRI scanner and an fMRI data acquisition system separate from the MRI scanner, comprising the steps of:
a) prompting an operator to input patient identification data into the system;
b) selecting an activation task with said system;
c) prompting the operator to check for proper patient positioning, equipment alignment and equipment operation with said system;
d) scanning to generate an anatomical MR image of the patient's brain with said MRI scanner;
e) presenting a series of stimuli to the patient using said system;
f) scanning to generate functional MR images of the patient's brain in synchronization with the presentation of said stimuli;
g) transferring said image data from said MRI scanner to said system;
h) verifying the integrity of the image data with said system; and
i) archiving said image and behavioral data to a removable media with said system.

2. The process of claim 1, further including the step of:
training the patient on an emulated task.

3. The process of claim 1, further including the step of:
checking patient response performance levels with respect to said stimuli, and warning the operator if minimum response levels are not detected.

4. The process of claim 1, wherein:
said step of verifying includes checking that the image data set is populated with expected amount of image data.

5. A process for acquiring task-activated fMRI patient data using an MRI scanner and an fMRI data acquisition module separate from the MRI scanner, comprising the steps of:
a) validating patient positioning and equipment alignment with said module;
b) training the patient on an emulated task with said module,
c) scanning an anatomical MR image of the patient's brain with said MRI scanner;
d) presenting a series of stimuli to the patient using said module;
e) checking for an normal level of patient responses to said stimuli; and
f) scanning for functional MR images in synchronization with the presentation of said stimuli with said MRI scanner.

6. The process of claim 5, further including the step of:
verifying the integrity of the image data with said module.

7. The process of claim 5, further including the step of:
transferring said image data from said MRI scanner to said module, and archiving said image data to a removable media.

8. The process of claim 5, further including the steps of:
prompting the operator to input patient identification data with said module, and selecting an activation task with said module.

9. The process of claim 8, further including the steps of:
verifying the integrity of the image data with said module,
transferring said image data from said MRI scanner to said module, and
archiving said image data to a removable medium.

10. An fMRI data acquisition module for acquiring task-activated fMRI patient data using an MRI scanner, comprising:
a) an fMRI control station including a data acquisition software program adapted for controlling said station for:
i) prompting the operator to input patient identification data with said module,
ii) prompting the operator to select a stimulation paradigm,
iii) prompting the operator to prepare the patient and equipment,
iv) presenting a series of stimuli to the patient in coordination with the performance of MRI scanning by said MRI scanner,
v) acquiring scanned image data from said MRI scanner, and
vi) verifying the integrity of the image data;
b) a presentation device connected to said control station for presenting sequences of stimuli to patients while they are in an MRI scanner; and
c) a patient response device connected to said control station.

11. The module of claim 10, wherein said software program also controls said station for:
emulating a stimulation paradigm for purposes of training patients for responding to stimulation paradigms.

12. The module of claim 10, wherein said operation of prompting to prepare the patient and equipment includes:
prompting the operator to check for proper patient positioning, equipment alignment and equipment operation.

13. The module of claim 10, wherein said response device includes:
four elongate keys having shallow depressions along their contact surfaces.

14. The module of claim 10, wherein said presentation device includes:
a video projector and a display screen.

15. The module of claim 14, further including a moveable cart of adjustable height for supporting and positioning said video projector, RF enclosure and display screen.
